(19) United States
(12) Patent Application Publication
(10) Pub. No.: US 2015/0216768 A1
(43) Pub. Date: Aug. 6, 2015

(54) SOLID SHAVING COMPOSITION

(71) Applicant: SHAV SHOWER BAR CORPORATION, Ottawa (CA)

(72) Inventors: Nicholas May, Ottawa (CA); Mario Merino, Mississauga (CA)

(21) Appl. No.: 14/428,744

(22) PCT Filed: Sep. 18, 2013
(86) PCT No.: PCT/CA2013/000781

§ 371 Date: Mar. 17, 2015

Related U.S. Application Data
(60) Provisional application No. 61/702,278, filed on Sep. 18, 2012.

(30) Foreign Application Priority Data
Nov. 19, 2012 (CA) 2795941

(51) Int. Cl. A61K 8/02; A61Q 9/02; A61K 8/92; A61K 8/36; A61K 8/34

(52) U.S. Cl. CPC A61K 8/0216; A61K 8/361; A61K 8/345; A61K 8/922; A61Q 9/02; A61K 2800/33

(57) ABSTRACT

A water-activable shaving composition is provided. The solid composition has a melting point between 41° C. +(0.2% +2.5° C.) and 46° C.+(0.2%+2.5° C.) when saturated with water. It is a propylene glycol based composition containing less than 15% by wt. of a fatty acid salt. The composition may also include water, glycerine, steareth 20, sodium stearate palmstar, polyethylene glycol powder, cocamide monoethanolamine (MEA), tea tree oil and optionally vegetable oil. The propylene glycol at least partly serves to both hydrate hairs prior to shaving as well as moisturize the skin.

### FIELD OF THE INVENTION

The invention relates to a shaving composition and more specifically a solid shaving composition.

### BACKGROUND

For conventional shaving with a safety razor or a straight razor, the area to be shaved is wetted and a shaving soap or foam is applied in order to more fully hydrate the hairs. This procedure is generally followed both by males, when shaving the face, and by females, when shaving legs, underarms, face, or bikini line and generally any area that is to be shaved.

Solid shaving compositions generally contain a significant portion of a soap base to help create hardness, lubrication and lather. Solid shaving compositions are generally used and applied with a brush to help create a foamy lather over the face. Afterwards, the area is shaved with a razor. This process requires the use of three instruments namely a brush, a solid shaving composition and a razor. This process is not ideal for shaving larger areas of the body, especially while in the shower. Soaps have been known to remove epidermal oils resulting in dry and desiccated skin.

One attempt has been made to combine a solid shaving composition around the razor to simplify the process. The solid shaving composition lubricates the skin while the razor shaves. However, incorporating a shaving solid around the razor increases the size of the shaving device. In an attempt to reduce the size of the shaving device contact area, typical attempts only incorporate a small quantity (about 1.5-10 g) of a shaving solid around the razor. As a result, when the user shaves, the solid shaving composition quickly erodes away. The shaving solid often erodes away long before the incorporated razor blade needs to be replaced. Attempts have been made to increase the longevity of the solid shaving composition and reduce its dissolution in hot water. These attempts include increasing the concentration of the soap and adding wear enhancers. This results in a solid shaving composition which can dry the skin and has a melting point above water temperatures comfortably used for showering or shaving.

One disadvantage associated with shaving soaps and foams is that the hydration of the hairs is not complete, and thus they often do not lubricate well at the razor edge. In addition, the soaps or foams tend to desiccate the skin, and creams or emollients must often be applied after shaving to re-hydrate the skin which can regardless be left irritated and/or dried.

In one prior art shaving system which attempts to increase hydration of the hairs, a 45% by volume 1,2-propylene glycol aqueous solution is first splashed on the area to be shaved. This helps to more fully hydrate the hairs. Then, after applying the 45% by volume 1,2-propylene glycol aqueous solution, a hot, wet towel is applied to the area to be shaved for one to two minutes in order to further increase hydration of the hairs. The 45% by volume 1,2-propylene glycol aqueous solution is then re-applied, followed by application of a shaving soap or foam. The area is then shaved. This procedure is time consuming, costly and requires a number of products to be present at time of shaving.

In some instances, for example in the military or when camping, dry shaving is necessary because one is limited in the amount of space or weight that be carried by the individual. Dry shaving generally is hard on the skin as it dries out the skin and results in nicks or cuts because the hairs have not been hydrated prior to shaving.

A need therefore exists for a solid shaving composition designed to melt at temperatures suitable for shaving, that hydrates hairs or generates a lubrication barrier over the skin on which the razor may glide prior to shaving which does not tend to desiccate the skin.

### SUMMARY OF THE INVENTION

The present invention relates to a solid shaving composition which is moulded into a hand held container, for example a stick form. The solid shaving composition is independent of a razor and is for direct application to the area to be shaved without the need of a brush for generating a lather. The user may apply the solid shaving composition onto dry or wet skin allowing the user to use a much smaller shaving device to remove the hairs from tight areas such as the bikini area. Considering that a hand held container can hold a large amount of a shaving solid (20-150 g), there is less emphasis on the rate at which the composition erodes away. Instead, an emphasis is made on the performance of the composition for the user.

In one non-limiting illustrative embodiment, there is provided a solid shaving composition which contains a reduced amount of soap (fatty acid salts) and has a melting temperature in the ideal range for shaving. One embodiment of a solid shaving composition is not soap based, rather it contains less than 15% by wt. of a fatty acid salt for gelling the propylene glycol and turning it into a solid. The solid shaving composition may have a melting point between 38° C. and 49° C.

In a further non-limiting illustrative embodiment, a water-activable shaving composition is provided. The solid shaving composition is formulated to have a melting point between 41° C.±(0.2%+2.5° C.) and 46° C.±(0.2%+2.5° C.) when saturated with water. The composition is a propylene glycol based composition comprising less than 15% by wt. fatty acid salts. Optionally, the composition may also include surfactants, chelating agents, antimicrobial agents, anti-inflammatory agents, emulsifiers, humectants, fragrance, colours, hair softeners, synthetic and/or natural preservatives, stabilizers, polymers and a combination of fore mentioned. The composition may comprise of water, alcohols, polyols, polyether diols, fatty acids, fatty acid amines and may optionally include terpinen-4-ol, α-terpinene, β-terpinene, 1,8-cineole and ethylenediaminetetraacetic acid salts. An example of these components include glycerine, steareth 20, sodium stearate palmstar, polyethylene glycol powder, cocamide monoethanolamine (MEA) and tea tree oil. The propylene glycol at least partly serves to both hydrate hairs prior to shaving as well as moisturize the skin. In addition, by dissolving a fatty acid salt powder into propylene glycol, the saponification step is avoided during the manufacturing of the composition. Further, different oils may be added into the product formulation further enhancing the feel of the product by reducing the desiccation of the skin. The composition may be prepared into a solid or cake form that allows application to the skin both with and without water. When water-activated and agitated over the skin, the composition provides a foamy lather that provides further lubrication for shaving. When water-activated and gently dispensed over the skin, the composition provides almost no lather.

In one non-limiting embodiment of the present invention, there is provided a solid shaving composition comprising less than 15% by wt. of fatty acid salt and having a melting point between about 38° C. and about 49° C. when saturated with water.

In a further embodiment to that outlined above, the composition is a propylene glycol based composition in that the major component or component present in the largest amount is propylene glycol.

In a further embodiment to that outlined above, the melting point is between about 41° and about 46° C. when saturated with water.

In a further embodiment to that outlined above, the composition further comprises between about 0.01 to about 3% W/W of EDTA in the form of disodium or trisodium EDTA.

In a further embodiment to that outlined above, the EDTA is present in an amount between about 0.1 to about 2% W/W.

In a further embodiment to that outlined above, the composition further comprises tea tree oil in an amount of from about 0.005 to about 0.8% W/W.

In a further embodiment to that outlined above, the composition further comprises:
water;
alcohol;
polyether;
fatty acid amine; and
fatty acid.

In a further embodiment to that outlined above, the alcohol is diol or polyol.

In a further embodiment to that outlined above, the fatty acid salt is in the form of sodium stearate palmstar.

In a further embodiment to that outlined above, the composition further comprises propylene glycol in an amount of least 50% W/W.

I another non-limiting embodiment, the present invention provides for a propylene glycol based shaving composition comprising less than 15% by wt. of fatty acid salt and having a melting point between about 38° C. and about 49° C. when saturated with water comprising:

| Raw material | % W/W |
|---|---|
| Propylene glycol | 50.000 |
| Glycerine | 5.000 |
| Steareth 20 | 2.200 |
| Sodium stearate palmstar (fatty acid salt) | 8.000 |
| Polyethylene glycol powder | 1.800 |
| Tea tree oil | 0.008 |
| EDTA | 0.5 |

In another non-limiting embodiment, the present invention provides for a razor comprising a solid shaving composition according to any of the embodiments outlined above, optionally in the form of a bar or tab incorporated into the razor.

### BRIEF DESCRIPTION OF THE DRAWINGS

FIG. 1 shows Table 4A outlining the effect of tea tree oil on microbial growth of bars exposed to warm tap water (Experiment 1);

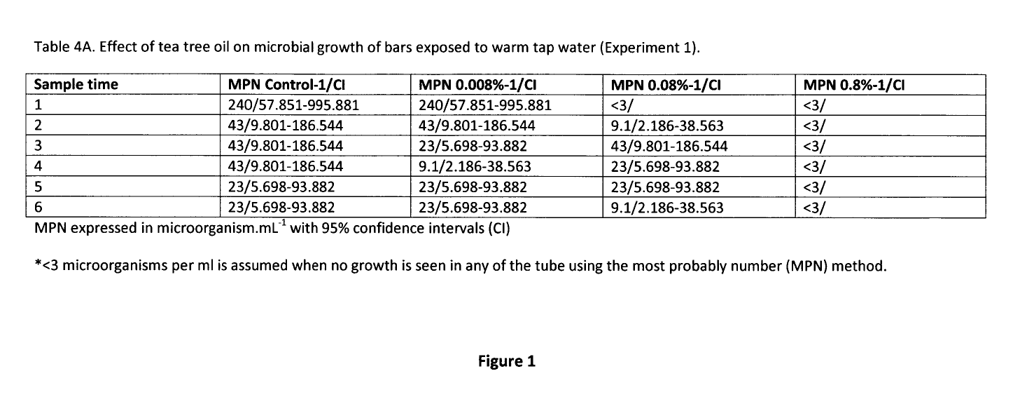

FIG. 2 shows Table 4B outlining the effect of tea tree oil on microbial growth of bars exposed to warm tap water (Experiment 2);

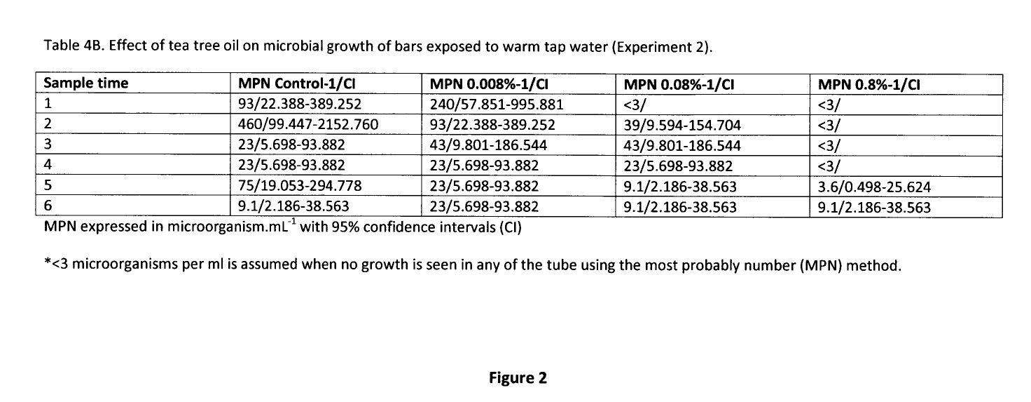

FIG. 3 shows Table 5A outlining the effect of tea tree oil on microbial growth of bars exposed to air (Experiment 1);

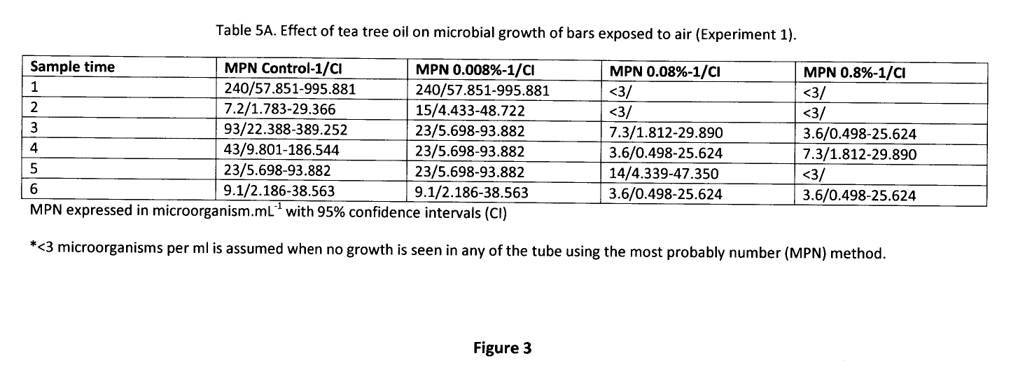

FIG. 4 shows Table 5B outlining the effect of tea tree oil on microbial growth of bars exposed to air (Experiment 2);

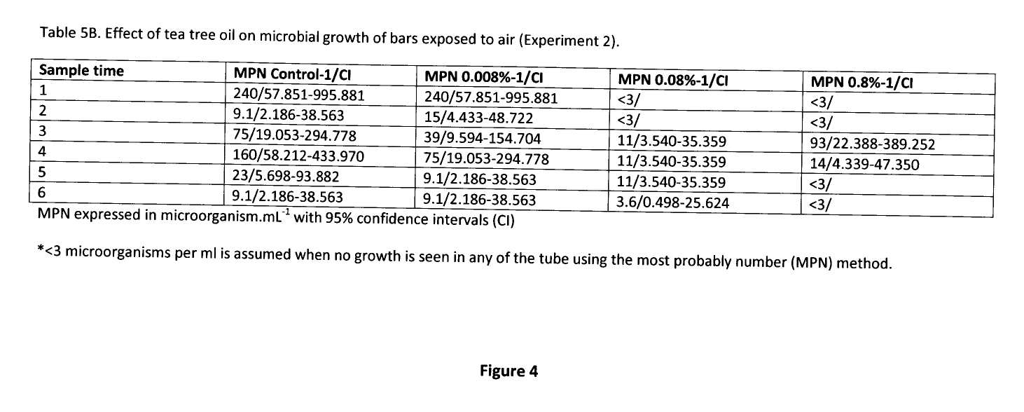

FIG. 5 shows Table 6A outlining the effect of tea tree oil on microbial growth in unopened bar over three weeks (Experiment 1);

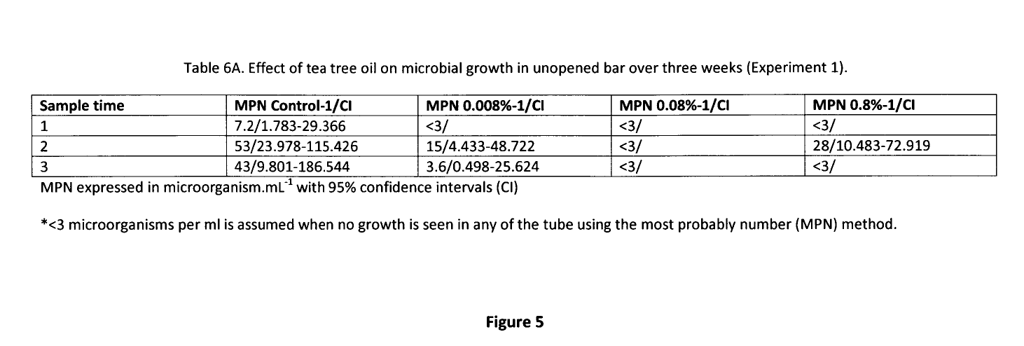

FIG. 6 shows Table 6B outlining the effect of tea tree oil on microbial growth in unopened bar over three weeks (Experiment 2);

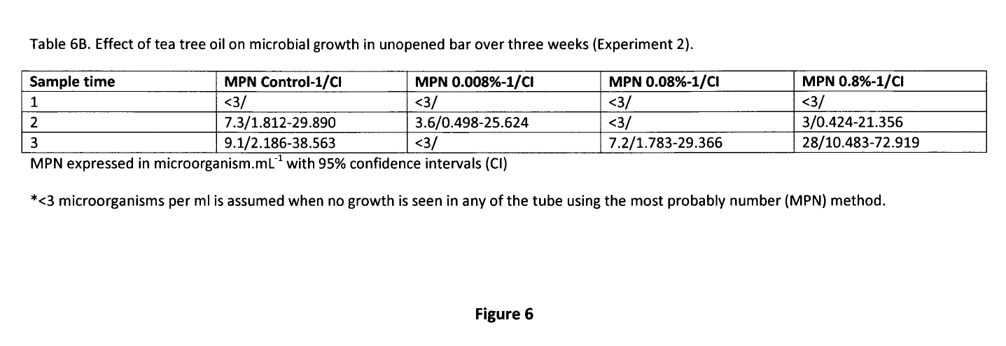

FIG. 7 shows Table 7A outlining the effect of tea tree oil on microbial growth of artificially inoculated Gram negative bacterium (Escherichia coli) (Experiment 1);

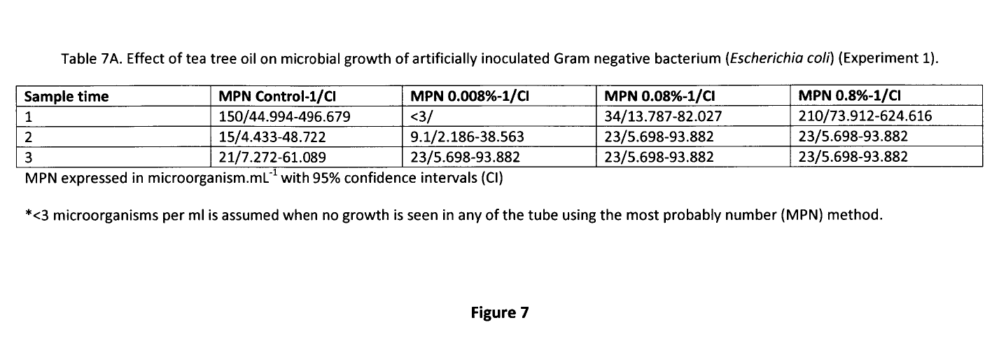

FIG. 8 shows Table 7B outlining the effect of tea tree oil on microbial growth of artificially inoculated Gram negative bacterium (Escherichia coli) (Experiment 2);

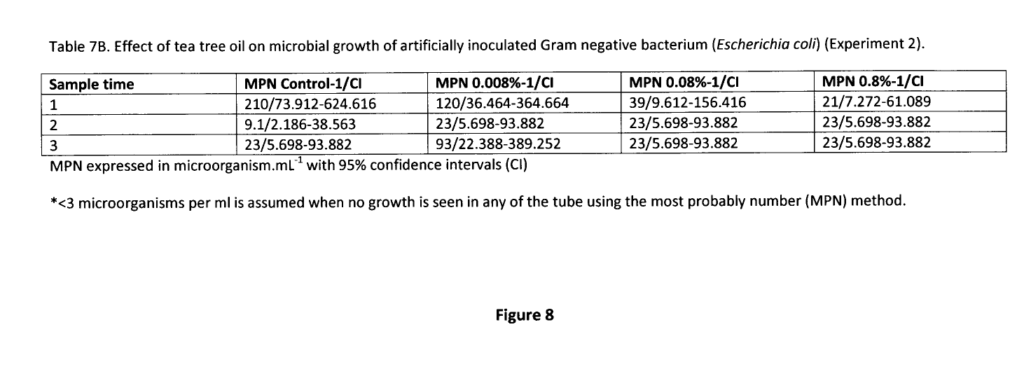

FIG. 9 shows Table 8A outlining the effect of tea tree oil on microbial growth of artificially inoculated Gram positive bacterium (Bacillus subtilis) (Experiment 1);

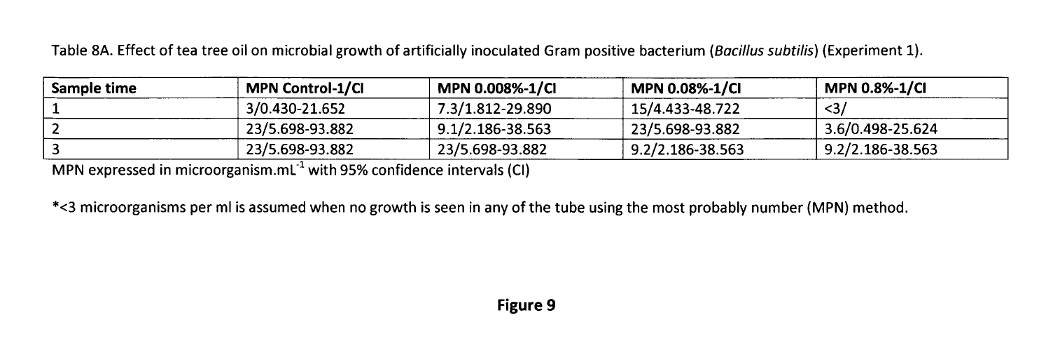

FIG. 10 shows Table 8B outlining the effect of tea tree oil on microbial growth of artificially inoculated Gram positive bacterium (Bacillus subtilis) (Experiment 2);

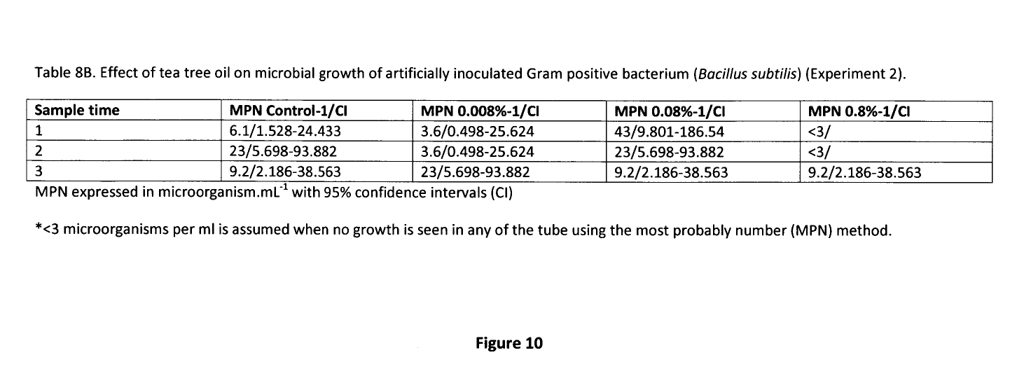

FIG. 11 shows Table 9A outlining the effect of tea tree oil on microbial growth of artificially inoculated yeast (Saccharomyces cerevisiae) (Experiment 1);

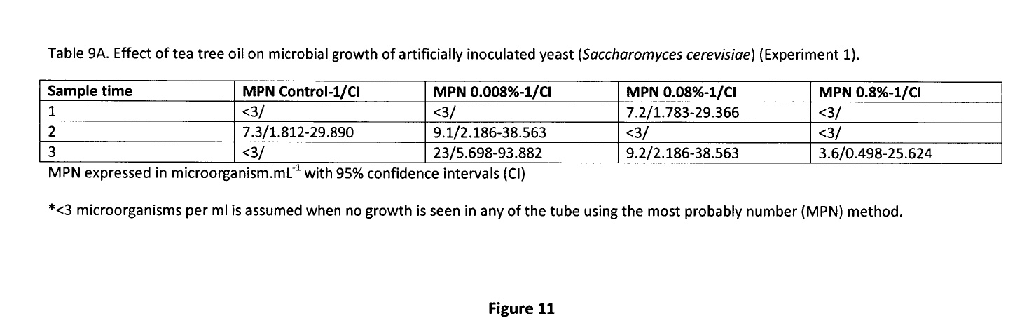

FIG. 12 shows Table 9B outlining the effect of tea tree oil on microbial growth of artificially inoculated yeast (Saccharomyces cerevisiae) (Experiment 2);

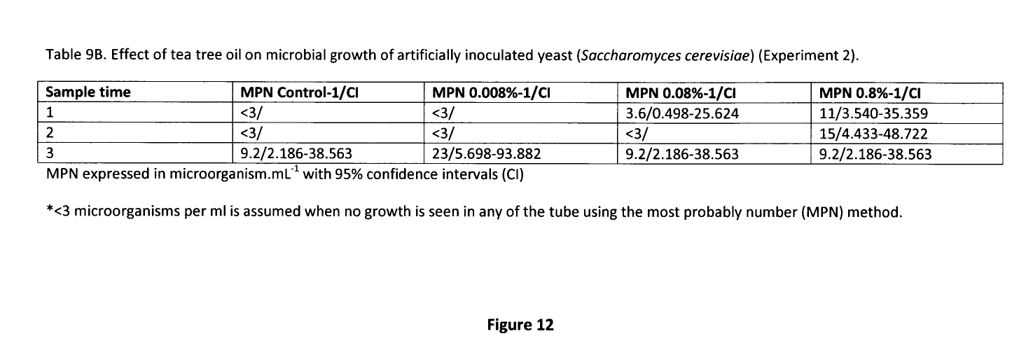

FIG. 13 shows Table 10A outlining the effect of Disodium EDTA and tea tree oil on microbial growth of artificially inoculated Gram negative bacterium (Escherichia coli) (Experiment 1); and

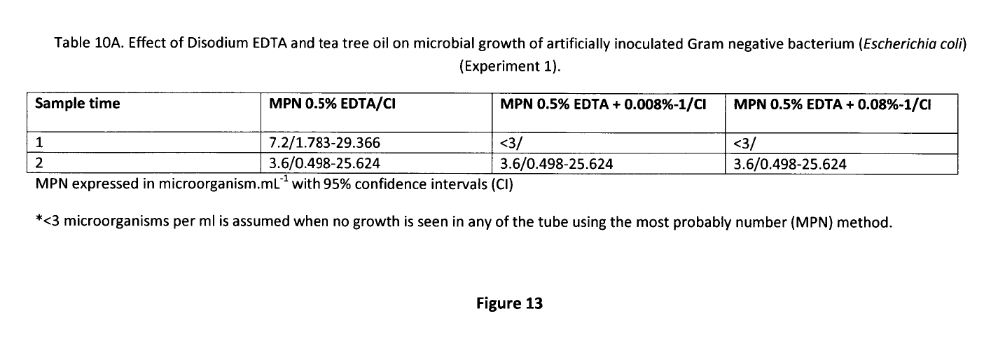

FIG. 14 shows Table 10B outlining the effect of Disodium EDTA and tea tree oil on microbial growth of artificially inoculated Gram negative bacterium (Escherichia coli) (Experiment 2).

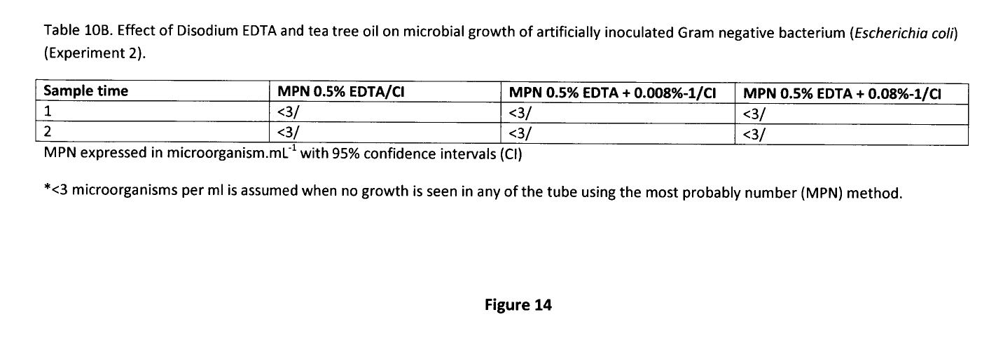

### DETAILED DESCRIPTION

A water-activatable composition for application to an area to be shaved is provided. The composition is not soap based but alternatively comprises propylene glycol as the major component of the composition.

In one illustrative non-limiting embodiment, an ideal solid shaving composition would melt and disperse over the skin at a temperature which is comfortable for shaving and would not desiccate the skin. The point at which a described solid shaving composition would melt when in contact with water would encourage its increased deposition over the area to be shaved. Increased deposition is ideal to help ensure the area to be shaved is well lubricated. In general, people prefer to shower or apply water onto their skin at a temperature slightly above their average body temperature of 37° C. In general, the water from a shower can range between 38° C. (cold) and 48° C. (scalding). In general, a shower is most comfortable between 39° C. and 46° C.

As the composition is not soap based, application thereof to the skin does not desiccate the skin and necessitate the use of a moisturizer following shaving. The propylene glycol at least partly serves to both hydrate and lubricate hairs prior to shaving as well as moisturize the skin. Without wishing to be limited by theory, it is believed that soap based shaving compositions strip a layer of oil from the skin thereby dehydrating or desiccating the skin. The propylene glycol composition is not soap based and therefore it is believed that it does not dehydrate or desiccate or at least mitigates dehydration or desiccation of the skin by avoiding the stripping oil from the skin.

By combining a fatty acid salt powder with propylene glycol, the saponification step is avoided during manufacturing of the composition. Therefore, compositions of the present invention may be quicker to manufacturer, may reduce extreme pH fluctuation, or may reduce degradation of process sensitive ingredients. In addition, the product may generally feels nicer on the skin. Because the composition contains less than 15% by wt. of a soap, the amount of oil removed from the skin during use may be reduced and thus desiccation of the skin is also reduced.

The propylene glycol based composition may be processed into a solid or cake for ease of storage and application to the skin. Upon application to the skin, the hairs to be shaved are hydrated and the skin at least partly moisturized. Water activation is not necessary before shaving, however, the application of water to the composition before or following application of the composition to the skin creates a lather that further results in lubrication of the skin and hairs and promotes easier and safer shaving.

The water-activatable composition comprises propylene glycol as the major component. As outlined above, the propylene glycol serves to both hydrate the skin and prevent desiccation of the skin while also allowing for a saponification step to be avoided during the manufacturing of the composition. The composition further comprises water and glycerine for moisturizing the skin. Steareth 20 is included in the composition to provide hardness to the composition for easier application to the skin and also adds a smooth characteristic to the cake for a more appealing feeling when applying to the skin. Sodium stearate palmstar allows the formation of the body of the cake or stick when combined with water, polyethylene glycol (more generically a polyoxyethylene) and propylene glycol. As indicated, the composition comprises polyethylene glycol powder used as a binder to help form the body of the cake or stick and provides lubrication for a smoother delivery of the product to the skin. Cocamide mea is used to lower the surface tension of the product when wet allowing for some foaming action to occur thereby giving the composition its water-activatable characteristic.

Tea tree oil may also be included in the composition to act as a preservative as well as a skin cleanser. Other preservatives may be included in addition to or as a substitute to tea tree oil such as DMDM hydantoin, methylchloroisothiazolinone and methylisothiazolinone, sodium benzoate, methyl paraben, propyl paraben, butyl paraben, trisodium EDTA and disodium EDTA for example. A fragrance may further be added to the composition, such as for example vanilla sugar or other or additional suitable fragrances. A combination of tea tree and vanilla may be used for example.

Hemp oil may also optionally be included in the composition as a source of oil rich in Omega fatty acids. Alternatively, avocado, coconut and/or olive oil may be used for example. In addition, various coloring additives may be included in the composition to obtain a desired color.

In one example that is not intended to be limiting, the composition comprises the components shown in Table 1.

TABLE 1
Raw Material for propylene glycol cake

| | | Quantity (kg) (to produce |
|---|---|---|
| Raw material | % W/W | 5000 units)) |
| Propylene glycol | 50.000 | 140.250 |
| D.I. water | 28.641 | 80.338 |
| Glycerine | 5.000 | 14.025 |
| Steareth 20 | 2.200 | 6.171 |
| Sodium stearate palmstar | 8.000 | 22.440 |
| Polyethylene glycol powder | 1.800 | 5.049 |
| Cocamide mea | 4.000 | 11.220 |
| Tea tree oil | 0.008 | 0.022 |
| Frangrance - Vanilla sugar | 0.300 | 0.842 |
| Hemp Oil | 0.001 | 0.003 |
| Color - Blue 1 | 0.010 | 0.028 |
| Color - Red 33 | 0.040 | 0.112 |

In another example, the composition may contain the following range of compounds as shown in Table 2.

TABLE 2
One example of the components of a water-
activatable shaving composition.

| Raw material | % W/W |
|---|---|
| Propylene glycol | 45-55 |
| D.I. water | 25-33 |
| Glycerine | 4-6 |
| Steareth 20 | 2-2.4 |
| Sodium stearate palmstar | 7-9 |
| Polyethylene glycol powder | 1.5-2.1 |
| Cocamide mea | 3-5 |

When water-activated and agitated over the skin, the composition provides a foamy lather that provides further lubrication for shaving. A foamy lather makes the composition ideal for shaving the face. When water-activated with large amounts of water, the composition provides a clear lubrication over the skin. A clear lubrication makes the composition ideal for shaving the body.

In addition, the melting point of the cake composition lowers when contacted with water as outlined in the experiment results shown in Table 3.

TABLE 3
Melting point results

| | Melting point range ° C. | With water ° C. |
|---|---|---|
| Sample number | ±(0.2% + 2.5° C.) | ±(0.2% + 2.5° C.) |
| 1 | 55-57 | 44-46 |
| 2 | 46-47 | 41-45 |
| 3 | 45-51 | 41-44 |
| 4 | 47-49 | 41-43 |

One non-limiting method of preparing the composition is as follows. The propylene glycol and water, preferably de-ionized water, are added together in a suitable tank and heated to between about 75-85° C. In order of addition, glycerine, steareth 20, sodium stearate palmstar, polyethylene glycol powder and cocamide mea are then added to the tank continuously mixing and allowing to dissolve before adding the next compound. The mixture is mixed and heated until the solution is substantially clear. Heating of the tank is then stopped and mixing is maintained. Once the solution has cooled to about 65° C., the tea tree oil, optional fragrance and optional colouring is added while mixing the solution. To avoid the product solidifying, the product is kept at 65° C. To form the cake or stick product, the container, for example an application stick container or mold, should be filled while the product is still in liquid form between about 60 and 65° C. to avoid hardening of the product before addition to the container or mold. The product may then be put through a first cooling cycle at about 39° C. for about 10 minutes to form the first crystals. The product may then be put through a second cooling cycle at about 50° C. for about 10 minutes to finalize the solidification of the product into the cake. It will be appreciated that the cooling cycles may be omitted and the product allow to cool naturally to room temperature to form the cake product, however, this will simply take longer. Alternatively, the product may be cooled before addition to the container or mold and simply cut from a larger cake for placing in the container or mold.

As the process does not include a saponification step, as is generally the case with soap based shaving products, the process may be carried out more quickly as the saponification reaction is omitted. In addition, saponification reactions generally include more extreme pH levels (typically a pH from 8-11) which can damage other additives in the composition and thus limit the type of additives one can use in the composition. The preparation of the present composition avoids the extreme basic or acidic pH levels observed in saponification and therefore does not expose the other additives to these pH levels thereby reducing the risk of damaging such additives. Generally, the process of prepared the presently disclosed composition fluctuates between a pH level of between 6-8.

The composition may be made into a cake for ease of application to a user as well as manufacturer into a portable product. Once in a cake form, the propylene glycol based composition may be incorporated into shaving products including razors, application sticks, hand held containers, without a container ect. It will be appreciated that any suitable form of the cake may be used for application to the skin prior to shaving to prepare the hairs for shaving and the stick, razor and hand held containers are merely non-limiting examples.

The composition may be applied in a number of different steps. For example, the composition may be pre-wetted and then applied to the area to be shaved to provide a lubricated, moisturized shaving area. Alternatively, the composition may be applied, without the need for pre-wetting, directly to the area to be shaved. The area with the composition may be simultaneously or subsequently exposed to water from a shower, bath, sink or other source. Upon application of water, the applied composition will become water-activated and provide lubrication. Alternatively, the composition may be applied to the area to be shaved with the pre- or post-application of water and a user may simply use the composition itself to prepare the area to be shaved. Although the area will be comparatively less lubricated, the composition will hydrate the area as well as moisturize the skin resulting in at least somewhat less discomfort during shaving. Such an application may be of use when water is not available or is inconvenient to carry or obtain such as when camping or for military use for soldiers operating in the field or with a limited source of water.

In addition to those compositions outlined above, EDTA may added to the composition as a preservative to inhibit the growth of for example E. coli. The addition of TTO may positively affect the inhibition produced by EDTA by acting in synergy to improve antimicrobial activity.

EDTA may be added as disodium EDTA. In a further embodiment, the EDTA may be trisodium EDTA. EDTA may be added in an amount of from about 0.01 to about 3% W/W. The range may be from about 0.1 to about 2% W/W. When an amount of about 3% W/W was added, whitening of the solid shave bar was observed.

Experimental

Evaluation of tea tree oil as an antimicrobial agent in solid shave gel bars.

Tea tree oil (TTO), the essential oil of Melaleuca alternifolia, is a natural antimicrobial agent. Terpinen-4-ol is the major component, making up approximately 30% of the oil, which exhibits strong antimicrobial and anti-inflammatory abilities. TTO also contains a-terpinene (5-13%), β-terpinene (10-28%) and 1,8-cineole (0-15%), which have been seen to act against bacteria and fungi (Carson, Hammer, & Riley, 2006: ISO 4730:2004). TTO compromises the structural and functional integrity of bacterial and fungal membranes by partitioning hydrocarbons into the membrane (Oyedemi, Okoh, Mabinya, Pirochenva, & Afolayn, 2009; Cox, et al., 2000). Most bacteria are susceptible to ≦1% TTO, but some bacterium found on skin such as staphylococci and micrococci can require ≧2%. Approximately ≦2% TTO concentrations have been found effective against most fungi (Carson, Hammer, & Riley, 2006).
Experiment 1: The exposure of shave gel bars containing 0%, 0.008%, 0.08% and 0.8% tea tree oil (TTO) to water and air as sources of microbial contamination.
Protocol 1: Water trial. Shave gel bars were made according to manufacturer's specifications with the exception of TTO concentration. Shave gel bars containing 0%, 0.008%, 0.08% and 0.8% TTO were exposed to 40° C. tap water for 10 minutes. After the 10 minute interval, water was drained off of the bars and the bars were placed into a 37° C. incubator. After 24 hours of incubation, samples were taken from the exposed bars and microbial levels were determined using the most probably number (MPN) method. Bars were exposed to water three times a week and tested twice each week for a period of three weeks. The experiment was repeated twice.
Results: In the first experiment (Table 4A), shave gel bars containing 0.8% TTO exhibited no microbial growth (approx. 100% inhibition) when exposed to water over the 3 week trial period. Shave gel bars containing 0.08% TTO had no microbial growth (approx. 100% inhibition) upon the initial sampling. The second sampling in week 1 showed 79% less microbial growth than the control bar without TTO. Other treatment TTO concentration and time period generally did not show antimicrobial activity. In the second experiment (Table 4B), 0.8% TTO exhibited approx. 100% inhibition of microbial growth over the first two weeks of the trial. The second sampling in week 1 showed 92% less microbial growth than the control bar without TTO. Other treatment TTO concentration and time period generally did not show antimicrobial activity.
Discussion and conclusions: 0.8% TTO is an effective concentration in which the growth of most microorganisms is inhibited. A concentration of 0.008% provides similar results to a concentration of 0% and therefore not effective in the inhibition of microbial growth. Shave gel bars containing 0.08% TTO again had no microbial growth (approx. 100% inhibition) upon the initial sampling, but loss antimicrobial activity over time.
Protocol 2: Air trial. Shave gel bars were made according to manufacturer's specifications with the exception of TTO concentration. Shave gel bars containing 0%, 0.008%, 0.08% and 0.8% TTO were placed on a bench top and exposed to air for 10 minutes. After 10 minutes, bars were resealed and placed into a 37° C. incubator. After 24 hours of incubation, samples were taken from the exposed bars and microbial levels were determined using the MPN method. The bars were exposed to air in the environment three times a week and tested twice a week for a period of three weeks.
Results: Shave gel bars containing 0.08 and 0.8% TTO exhibited zero to very low levels of microbial growth when exposed to air over a 3-week trial period. Inhibition of growth ranged from 60-100% at these two concentrations an in both experiments when compared to the controls (Table 5A and 5B).
Discussion and conclusions: 0.08 and 0.8% TTO are effective concentrations in which the growth of many microorganisms is inhibited. A concentration of 0.008% TTO provides similar results to a concentration of 0% TTO and therefore not effective in the inhibition of microbial growth.
Experiment 2: Evaluation of the potential for microbial growth in unopened bars (microbial shelf life).
Protocol 1: Shave gel bars made according to the manufacturer's specifications with the exception of TTO content. Shave gel bars containing 0%, 0.008%, 0.08%, and 0.8% TTO were made and placed in a 37° C. incubator. Samples were taken from unopened bars once a week for three weeks after manufacturing of the bars. Microbial levels were determined using the MPN method. The experiment was repeated twice.
Results: Shave gel bars containing 0.08% and 0.8% TTO showed zero to very low level of microbial growth. In some cases this was also seen amongst the bars containing 0.008% TTO (Tables 6A and 6B).
Discussion and conclusions: Although bars are left unopened on store shelves there still lies the risk for initial contamination and growth of microorganisms. With no TTO present in the bars, low levels of growth were seen on the bars. With TTO present, at all three concentrations showed no to very little growth compared to the control without TTO.
Experiment 3: Evaluation of the sustainability of growth of Escherichia coli (Gram negative bacterium), Bacillus subtilis (Gram positive bacterium), and Saccharomyces cerevisiae (yeast) on shave gel bars containing 0%, 0.008%, 0.08% and 0.8% TTO.
Protocol 1: Shave gel bars were made according to manufacturer's specifications with 0%, 0.008%, 0.08% and 0.8% TTO. Bars with a surface area of 17.72 cm were individually artificially inoculated with 106 cells of E. coli, B. subtilis or S. cerevisiae. Inoculated bars were left to incubate for 48 hours at 37° C. before being sampled. After 48 hours, bars were sampled and the microbial levels were determined using the MPN method. Sampling was completed once a week for three weeks. The experiment was repeated twice.
Results: No complete inhibition of E. coli growth was seen on any of the bars for the three week trial duration in both experiments (Table 7A and 7B). However, those containing 0.08 or 0.8% TTO exhibited less growth (77-90% inhibition when compared to the control) at the first sampling period (1 week) in both experiments with artificial inoculations of E. coli.

Bars containing 0.8% TTO effectively reduced (over the entire trial period) and in some instances halted the growth (for up to 2 weeks) of B. subtilis (Tables 8A and 8B).

In all cases, bars treated with S. cerevisiae exhibited virtually no growth to very little growth in all tested treatments (Tables 9A and 9B).
Discussion and conclusions: complete or partial inhibition of E. coli and B. subtilis growth was found with 0.08-0.8% TTO. TTO was not necessary to inhibit S. cerevisiae as little growth was found on the control treatments without TTO.
Experiment 4: Evaluation of the sustainability of growth of E. coli on shave gel bars containing 0.5% Disodium EDTA (EDTA), 0.5% EDTA with 0.008% TTO, and 0.5% EDTA with 0.08% TTO.
Protocol 1: Shave gel bars were made according to manufacturer's specifications with 0.5% EDTA, 0.5% EDTA with 0.008% TTO, and 0.5% EDTA with 0.08% TTO. Bars with a surface area of 17.72 cm were inoculated with 106 cells of E. coli. Inoculated bars were left to incubate for 48 hours at 37° C. before being sampled. After 48 hours, bars were sampled and the microbial levels were determined using the MPN method. Sampling was completed once a week for two weeks. The experiment was repeated twice.
Results: Virtually no growth to very little growth was seen amongst all bars containing EDTA and those containing EDTA with TTO (Table 10A and 10B). Bar containing both EDTA and TTO were more active than bars with EDTA alone in experiment 1.
Discussion and conclusions: The presence of EDTA alone was sufficient to strongly inhibit the growth of E. coli. The addition of TTO may positively affect the inhibition produced by EDTA by acting in synergy to improve antimicrobial activity.

It will be appreciated by persons skilled in the art that various modifications and/or variations may be made to the embodiments of the invention without departing from the scope or spirit of the invention. The embodiments disclosed herein are therefore intended to be illustrative only and are not intended to be limiting in any way.

1. A solid shaving composition comprising up to 15% by wt. of fatty acid salt and having a melting point between about 38° C. and about 49° C. when saturated with water.

2. The solid shaving composition according to claim 1, wherein the composition is a propylene glycol based composition.

3. The solid shaving composition according to claim 1, wherein the melting point is between about 41° C. and about 46° C. when saturated with water.

4. The solid shaving composition according to claim 1, further comprising between about 0.01 to about 3% W/W of EDTA in the form of disodium or trisodium EDTA.

5. The solid shaving composition according to claim 4, wherein the EDTA is present in an amount between about 0.1 to about 2% W/W.

6. The solid shaving composition according to claim 1, further comprising tea tree oil in an amount of from about 0.005 to about 0.8% W/W.

7. The solid shaving composition according to claim 4, further comprising tea tree oil in an amount of from about 0.005 to about 0.8% W/W.

8. The solid shaving composition according to claim 4, further comprising:
water;
alcohol;
polyether;
fatty acid amine; and
fatty acid.

9. The solid shaving composition according to claim 8, wherein the alcohol is diol or polyol.

10. The solid shaving composition according to claim 1, wherein the fatty acid salt is in the form of sodium stearate palmstar.

11. The solid shaving composition of claim 1, further comprising propylene glycol in an amount of least 50% W/W.

12. A propylene glycol based shaving composition comprising up to 15% by wt. of fatty acid salt and having a melting point between about 38° C. and about 49° C. when saturated with water comprising:

| Raw material | % W/W |
|---|---|
| Propylene glycol | 50.000 |
| Glycerine | 5.000 |
| Steareth 20 | 2.200 |
| Sodium stearate palmstar (fatty acid salt) | 8.000 |
| Polyethylene glycol powder | 1.800 |
| Tea tree oil | 0.008 |
| EDTA | 0.5 |

13. A razor comprising the solid shaving composition according to claim 1.

14. A razor comprising the solid shaving composition according to claim 4.

15. (canceled)

16. The solid shaving composition of claim 1, further comprising Steareth 20.
